(19) United States
(12) Patent Application Publication
(10) Pub. No.: US 2026/0115195 A1
(43) Pub. Date: Apr. 30, 2026

(54) CAFFEINE SUSTAINED RELEASE FORMULATION

(71) Applicant: Prolevi Bio AB, Lund (SE)

(72) Inventors: Sahil Gupta, Idstein (DE); Daniel Bar-Shalom, Odense C (DK)

(21) Appl. No.: 19/366,832

(22) Filed: Oct. 23, 2025

(30) Foreign Application Priority Data
Oct. 25, 2024 (EP) EP 24208919.1

(51) Int. Cl. A61K 31/522; A61K 9/20; A61K 9/28

(52) U.S. Cl. CPC A61K 31/522; A61K 9/2013; A61K 9/2054; A61K 9/2095; A61K 9/282; A61K 9/2846; A61K 9/2893

(57) ABSTRACT

The present disclosure relates to the field of sustained release compositions of caffeine and uses thereof. The formulations according to the present disclosure accommodate for caffeine sustained release.

### TECHNICAL FIELD

The present disclosure relates to the field of sustained release compositions of caffeine and uses thereof. The formulations according to the present disclosure accommodate for caffeine sustained release.

### BACKGROUND

Caffeine (1,3,7-trimethylxanthine) is found in beverages such as coffee, tea and soda as well as certain foods such as chocolate and cocoa, and is well reported to improve cognitive performance, energy, and mental status. Caffeine exerts its main effects by blocking adenosine receptors and following low to moderate doses caffeine doses, alertness, vigilance, attention, reaction time and attention improve. In addition, caffeine has ergogenic properties with a positive impact in diverse types of exercise. Thus, caffeine is a commonly used, safe compound that may have a beneficial effect in occupational or leisure activities benefiting from cognitive and physical improvements (Mclellan et al. 2016).

Caffeine is rapidly absorbed and achieves peak plasma concentrations rapidly followed by a steep decline (McLellan et al. 2016). This steep decline is sometimes associated with caffeine “crashes” which present as a feeling of exhaustion and tiredness with a fast onset.

Often, repeat intake of caffeine is needed to ensure its beneficial effects. This is not only inconvenient but can also result in the build-up of high levels of caffeine, which have been associated with negative effects such as sleep disorders, feeling of nervousness, headaches, light-headedness and “jitters” (Herrick et al. 2007).

Thus, there is a strong need for caffeine products that can provide a convenient administration of caffeine that achieves the beneficial effects while minimizing or off-setting downsides associated with caffeine consumption.

### SUMMARY

The present invention provides one such solution to the above-mentioned problem, by providing formulations that produce a sustained release of caffeine, and thereby achieve a controlled presence of caffeine in plasma for extended periods while diminishing peak plasma concentrations of caffeine, as shown in the Examples

The sustained-release caffeine formulations of the present disclosure provide the ability to achieve the beneficial effects of caffeine while reducing the number of repeated doses required compared to traditional sources of caffeine. Thus, offering an effective and convenient alternative to repeated daily doses of caffeine required to achieve a satisfactory stimulant effects e.g. enhanced alertness, reaction performance, improved vigilance, and cognitive functions throughout the day while avoiding drawbacks like the symptoms of a caffeine crash, jitteriness and tiredness.

Such advantages could be useful in daily activities requiring extra attentiveness and when access to continuous intake of immediate-release caffeine is limited. Or in specific circumstances like occupational activity during nighttime or jet-lag.

Thus, in one aspect, the present disclosure provides a composition comprising:
(a) caffeine, or a pharmaceutically acceptable salt thereof, or food safe salt thereof;
(b) hydroxypropyl methyl cellulose acetate succinate (HPMC-AS) and/or hydroxypropyl methyl cellulose (HPMC), and
(c) polyethylene oxide (PEO);
   wherein the weight ratio between PEO (c) and the HPMC and/or HPMC-AS (b) is of 8:2 to 9:0.1.

In one aspect the present disclosure provides a method of manufacturing a composition comprising the steps of:
(a) preparing a melt uniform dispersion comprising i) PEO, ii) hydroxypropyl methyl cellulose and/or HPMC-AS and iii) caffeine, or a pharmaceutically acceptable salt thereof, and
(b) cooling the melt to obtain a solid composition.

In one aspect the present disclosure provides a method of manufacturing a composition comprising the steps of:
(a) mixing powders of caffeine or a pharmaceutically acceptable salt thereof, HPMC and/or HPMC-AS; and PEO, and
(b) compacting the mixture to obtain uniform dispersion of components in a matrix.

In one aspect the present disclosure provides a composition obtained by the method descried herein. As shown in the Examples, the compositions according to the present disclosure provide for a decreased Cₘₐₓ of caffeine, compared to immediate release formulations, while providing the equivalent total exposure of caffeine. This highlights the ability of the compositions to extend the beneficial effects of caffeine while avoiding undesirable side effects due to excessive caffeine build-up.

Thus, in another aspect the present disclosure relates to a method for obtaining a tₘₐₓ of caffeine between 4 h and 9 h in a subject, such as 5 h, such as 6 h, such as 7 h, such as 8 h caffeine in a subject, said method comprising administering the composition described herein.

In one aspect, the present disclosure relates to a method for obtaining Cₘₐₓ of caffeine of between 0.5 and 4 mg/mL in a subject, such as 1 mg/mL, such as 1.5 mg/mL, such as 2 mg/mL, such as 2.5 mg/mL, such as 3 mg/mL, such as 3.5 mg/mL caffeine in a subject, said method comprising administering the composition described herein.

### DESCRIPTION OF DRAWINGS

FIG. 1: Dissolution profiles of 100 mg immediate release (IR, top line) caffeine tablet vs sustained release (SR, bottom line) tablets according to the present disclosure. Further details in example 1.

FIG. 2: Concentration of caffeine absorbed in from immediate release tablets in humans (light grey, solid line) and predicted by in vitro in vivo correlation (IVIVC) (light grey, discontinuous line) and from sustained release tablets in humans (dark grey, solid line) and predicted by IVIVC (dark grey, discontinuous line). Further details in example 1 and 2.

### DEFINITIONS

“Cₘₐₓ” is a term used in pharmacokinetics to refer to the maximum (or peak) concentration that a drug achieves in a specified compartment or test area of the body (e.g. blood, serum, or plasma) after the drug has been administrated and prior to the administration of a second dose.

It has been shown caffeine Cₘₐₓ in plasma strongly correlates with Cₘₐₓ of caffeine measured in saliva of human subjects (Perera et al. 2011). Thus, the Cₘₐₓ in plasma may be estimated or assumed to be equivalent to the Cₘₐₓ determined in saliva.

“tₘₐₓ” is the term used in pharmacokinetics to describe the time at which the Cₘₐₓ is observed.

It has been shown that caffeine tₘₐₓ in plasma strongly correlates with tₘₐₓ of caffeine measure in saliva of human subjects (Perera et al. 2011). Thus, the tₘₐₓ in plasma may be estimated or assumed to be equivalent to the tₘₐₓ determined in saliva.

“Half-life (t_{1/2})” is a term that refers to the time required for half of the drug in blood to be eliminated.

“Apparent volume of distribution (Vd)” or “Apparent volume of drug distribution” is a parameter calculated from amount of drug administered and its observed concentration (mass/volume) for an individual with given body mass.

“Oral bioavailability” refers to the fraction of orally administered drug available in the blood calculated relative to drug concentration after intravenous injection.

“Sustained release” is a term used to refer to a mechanism used in compositions or formulations to provide release of a drug or an active pharmaceutical ingredient from the composition or formulation over an extended period time to sustain plasma levels, as opposed to all at once, or as opposed to an immediate release or a burst release. In some embodiments, as used herein, the terms “sustained release”, “extended release”, “controlled release” and “modified release” are interchangeable to refer to this mechanism.

In some embodiments, as used herein, the terms “composition” and “formulation” are used interchangeably.

The term “about” as used herein to refer to an amount or percentage is to be interpreted as a variation of +10% with respect the value of the amount or percentage it refers to, such as +5%.

“Matrix” as used herein refers to a well-mixed homogeneous mixture of one or more excipients, particularly polymers, wherein one or more active ingredients are homogeneously dispersed.

### DETAILED DESCRIPTION

The present disclosure relates to the field of sustained release compositions of caffeine (1,3,7-trimethylxanthine, Formula I), and uses thereof. The formulations according to the present disclosure accommodate for caffeine release over an extended period.

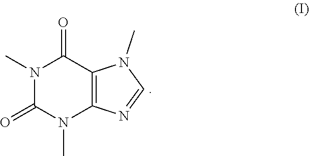

Thus, one aspect of the present disclosure relates to compositions comprising:
(a) caffeine, or a pharmaceutically acceptable salt thereof,
(b) hydroxypropyl methyl cellulose acetate succinate (HPMC-AS) and/or hydroxypropyl methyl cellulose (HPMC), and
(c) polyethylene oxide (PEO).

The inventors have surprisingly shown that the compositions disclosed herein provide for a sustained dissolution rate and release profile of caffeine with a safe return to baseline.

This is advantageous for caffeine release, as the steep decline in caffeine levels following regular or immediate release caffeine intake is avoided with a sustained release. Another advantage of a sustained caffeine release is that the risk of build-up of high levels of caffeine following repeated intake of immediate release caffeine is minimized or avoided.

Thus, the present disclosure relates to caffeine products, particularly tablets, that can provide a convenient administration of caffeine that achieves the beneficial effects while minimizing or off-setting downsides associated with caffeine consumption.

The advantages of the formulations and compositions disclosed herein are numerous and include:
reduced number of doses required and simplified administration compared to immediate release formulations,
reduced undesirable side effects compared to immediate release formulations, and/or
Extended time of beneficial stimulant effects.

### Composition

Sustained or controlled release technology is a collection of mechanisms, such as erosion and diffusion, used in compositions or formulations to slowly dissolve and release or slowly release and dissolve a substance over time. Extended-release formulations may be taken less frequently than immediate-release formulations, and they usually keep steadier levels of the active ingredient in the bloodstream.

The inventors have shown that compositions comprising an active ingredient, caffeine, in a matrix comprising: hydroxypropyl methyl cellulose (HPMC) and/or hydroxypropyl methyl cellulose acetate succinate (HPMC-AS) and polyethylene oxide (PEO); provide sustained release of caffeine with a desirable profile for caffeine over an extended period of time with a safe return to base line.

Thus, in one embodiment the present disclosure relates to a pharmaceutical composition comprising:
(a) caffeine, or a pharmaceutically acceptable salt thereof,
(b) hydroxypropyl methyl cellulose acetate succinate (HPMC-AS) and/or hydroxypropyl methyl cellulose (HPMC), and
(c) polyethylene oxide (PEO);
   wherein the weight ratio between PEO (c) and the HPMC and/or HPMC-AS (b) is of 8:2 to 9:0.1.

The inventors have shown that pharmaceutical compositions comprising PEO, HPMC and/or HPMC-AS in specific amounts provide sustained release of caffeine a desirable profile over an extended period of time.

The caffeine may be in one embodiment be anhydrous caffeine. In one embodiment, the composition comprises a pharmaceutically acceptable salt of caffeine, or a food safe salt of caffeine. Any food safe or pharmaceutically acceptable salt of caffeine is considered within the present disclosure.

Polyethylene oxide (PEO) is obtained by polymerization of ethylene oxide. PEO may also be referred to as polyethylene glycol (PEG) In this text, terms “PEG” and “PEO” are used interchangeably.

HPMC (CAS number: 9004-65-3) and HPMC-AS (CAS number: 71138-97-1) are cellulose derivatives wherein some free hydroxyl groups in cellulose have been substituted with hydroxypropyl and methyl groups. In the case of HPMC-AS, further hydroxyl groups are substituted with acetate (acetyl) and succinate (succinyl) groups. Generally, these materials are used as coatings to delay the release of a medicinal compound into the digestive tract. Surprisingly, the inventors have found that introducing HPMC and HPMC-AS as part of the matrix provides for an advantageous and robust mechanism of release based on erosion. This erosion-based release advantageously provides a more stable and consistent release compared to diffusion-based or osmotic-based release mechanisms.

In one embodiment, the weight ratio between PEO (component c) and the HPMC and/or HPMC-AS (component b) is of 8:2 to 9:0.1. In one embodiment the weight ratio between PEO (component c) and the HPMC and/or HPMC-AS (component b) is between about 8:1 and about 9.5:1, such as 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1 or 9.5:1. In one embodiment, the weight ratio between PEO (component c) and the HPMC and/or HPMC-AS (component b) is between about 8:2, such as 9:1, 9.5:1, 9:0.5, 8.5:1 or 8.6:1.

Reference to component b is to HPMC, HPMC-AS, or a mixture of HPMC and HPMC-AS as described herein.

In one embodiment, the weight ratio between PEO (component c) and the HPMC and/or HPMC-AS (component b) is of 8:2 to 9:1. In one embodiment, the weight ratio between PEO (component c) and the HPMC and/or HPMC-AS (component b) is of 9:1 to 9.5:0.5.

In one embodiment, the weight ratio between PEO (component c) and the HPMC and/or HPMC-AS (component b) is of 8.5:1.5 to 9.5:0.5. In one embodiment, the weight ratio between PEO (component c) and the HPMC and/or HPMC-AS (component b) is of 8:2 to 9.5:0.5.

In one embodiment, the weight ratio between PEO (component c) and the HPMC and/or HPMC-AS (component b) is of 8:2. In one embodiment, the weight ratio between PEO (component c) and the HPMC and/or HPMC-AS (component b) is of 9:1.

For example, a weight ratio between PEO (component c) and HPMC and/or HPMC-AS (component b) of 9:1 refers to a ratio of 9 parts by weight of PEO to 1 part by weight of HPMC, or HPMC-AS, or the combination of HPMC and HPMC-AS.

In one embodiment, caffeine, or a pharmaceutically acceptable salt thereof, or food safe salt thereof, is present in an amount equal or less than 50% by weight of the composition, such as below 40%, such as below 30%, such as below 20%, such as below 10%, such as below 5% by weight of the composition. In one embodiment, caffeine, or a pharmaceutically acceptable salt thereof, is present in an amount between 50% and 5% by weight of the composition, such as between 40% and 5%, such as between 35% and 5%, such as between 30% and 5%, such as between 30% and 10%, such as between 30% and 15%, such as between 20% and 15% by weight of the composition. In one embodiment, the caffeine, or a pharmaceutically acceptable salt thereof, is present in an amount between 50% and 30% by weight of the composition, such as between 50% and 35%, such as between 50% and 40%, such as between 50% and 45% by weight of the composition.

In one embodiment, caffeine, or a pharmaceutically acceptable salt thereof, is present in an amount of 50%, 45%, 40%, 35%, 30%, 25%, 20%, 15%, 10%, or 5% by weight of the composition.

In one embodiment, caffeine, or a pharmaceutically acceptable salt thereof, is preferably present in an amount of 30% to 5% by weight. In one embodiment, caffeine, or a pharmaceutically acceptable salt thereof, is more preferably present in an amount of 30% to 10% by weight.

In one embodiment, caffeine, or a pharmaceutically acceptable salt thereof, is present in an amount of 20% by weight of the composition.

In one embodiment, HPMC-AS is present in an amount of about 1% to 20% by weight of the composition, such as about 2% to 20%, such as about 5% to 15%, such as about 6% to 14%, such as about 7% to 13%, such as about 8% to 12%, for example about 8%.

In one embodiment, HPMC-AS is present in an amount of about 3% to 20% by weight of the composition. In one embodiment, HPMC-AS is present in an amount from 12% to 20% by weight of the composition. In one embodiment, HPMC-AS is present in an amount from 6% to 12% by weight of the composition. In one embodiment, HPMC-AS is present in an amount from 14% to 20% by weight of the composition.

In one embodiment, HPMC-AS is present in an amount of about 8%.

In one embodiment, the composition does not comprise HPMC, for example wherein the composition is substantially free of HPMC.

In one embodiment, hydroxypropyl methyl cellulose (HPMC) is present in an amount of about 1% to 20%, such as about 2% to 20%, such as about 5% to 15%, such as about 6% to 14%, such as about 7% to 13%, such as about 8% to 12%, for example about 8%.

In one embodiment, HPMC is present in an amount of about 3% to 20% by weight of the composition. In one embodiment, HPMC is present in an amount from 12% to 20% by weight of the composition. In one embodiment, HPMC is present in an amount from 6% to 12% by weight of the composition. In one embodiment, HPMC is present in an amount from 14% to 20% by weight of the composition.

In one embodiment, HPMC is present in an amount of about 8%.

In one embodiment, the composition does not comprise HPMC-AS, for example wherein the composition is substantially free of HPMC-AS.

In one embodiment, HPMC-AS and/or hydroxypropyl methyl cellulose is present in a total amount of about 1% to 10%, such as about 2% to 20%, such as about 5% to 15%, such as about 6% to 14%, such as about 7% to 13%, such as about 8% to 12%, for example about 8%.

In one embodiment, HPMC-AS and/or HPMC is present in an amount of about 3% to 20% by weight of the composition. In one embodiment, HPMC-AS and/or HPMC is present in an amount from 12% to 20% by weight of the composition. In one embodiment, HPMC-AS and/or HPMC is present in an amount from 6% to 12% by weight of the composition. In one embodiment, HPMC-AS and/or HPMC is present in an amount from 14% to 20% by weight of the composition.

In one embodiment, HPMC-AS and/or hydroxypropyl methyl cellulose is present in an amount of about 8%.

In some embodiments, the HPMC-AS is characterized by viscosity of 1 to 100 mPa·s when measured under standard conditions (2% HPMC-AS w/w in 10% NaOH solution at 20° C.); preferably a viscosity of 1 to 10 mPa·s, more preferably a viscosity of 2 to 4, or 3 mPa's when measured under standard conditions (2% HPMC-AS w/w in 10% NaOH solution at 20° C.).

In some embodiments, the HPMC-AS is characterized by;
a) an acetyl substitution content of 5% to 14%, such as an acetyl substitution content of 5% to 9%, or 7% to 11%, or 10% to 14%;
b) an succinyl substitution content of 8% to 18%, such as a succinyl substitution content of 4% to 8%, 10% to 14%, or 14% to 18%;
c) a methoxy substitution content of 20% to 26%, such as a methoxy substitution content of 20% to 24%, 21% to 25%, or 22% to 26%; and/or
d) a hydroxypropyl substitution content 5% to 10%, such a hydroxypropyl substitution content of 5% to 9%, or 6% to 10%.

Additionally, the inventors have surprisingly shown that compositions comprising HPMC-AS mixed in a matrix, instead of as a coating or encapsulation, provide a sustained release profile with pharmacokinetic properties favorable for caffeine. Furthermore, due to functional groups present in HPMC-AS, this component further provides improved protection to the active ingredient through diverse and varying conditions in the gastrointestinal track, especially pH.

In one embodiment, PEO has an average molecular weight between 10 kDa to 2000 kDa, such as 50 kDa to 1000 kDa, such as 100 kDa to 500 kDa, such as 150 kDa to 300 kDa.

In one embodiment, PEO is present in an amount of about 50 to 99%, such as about 65% to 95% by weight of the composition, such as about 60% to 90%, such as about 75% to 85%, such as about 70% to 80%, such as about 72%. In one embodiment, PEO is present in an amount of about 66 to 76%, such as 66% to 67%, such as 67% to 68%, such as 68% to 69%, such as 69% to 70%, such as 70% to 71%, such as 71% to 72%, such as 72% to 73%, such as 73% to 74%, such as 74% to 75%, such as 75% to 76%.

In one embodiment, PEO is present in an amount of about 72% by weight of the composition.

In one embodiment, the components a) (caffeine), b) (HPMC and/or HPMC-AS) and c) (PEO) are in a single matrix.

In one embodiment, the matrix is a uniform dispersion of caffeine, or a pharmaceutically acceptable salt thereof; the HPMC and/or HPMC-AS; and the PEO, in amounts as described herein.

“Uniform dispersion” means one wherein the components are evenly or homogeneous distributed through the dispersion.

In one embodiment, the present disclosure provides for a composition comprising a matrix, said matrix comprising:
i. caffeine or a pharmaceutically acceptable salt thereof as described herein,
ii. HPMC and/or HPMC-AS; and
iii. polyethylene oxide (PEO), wherein the weight ratio of PEO (component i.) to HPMC and/or HPMC-AS (component ii.) is between 8:2 and 9:0.1.

PEO, HPMC and HPMC-AS are regarded as hydrophilic and gel forming. In water, a matrix made up of any of them or mixtures thereof will experience a series of processes leading to dissolution: water penetration, hydration, disentanglement, gel formation and migration of loose chains into the media.

In cases where the matrix is produced in such a way as to prevent fast penetration of water, the first step, the result is that the dosage unit is gradually eroded only forming a thin release layer on the surface. Such a matrix can be achieved by thermoplastic processes such as injection molding, hot-melt extrusion, calendering or by compression at sufficient pressures due to the high deformability/plasticity and low melting point of, in particular PEO. The object is to prevent water penetration by reducing the occurrence of cracks or inter-particular channels.

The matrix may be obtained by direct compression of a mixture of powders of i., ii., and iii due to the physical properties of PEO.

The matrix according to the present disclosure releases caffeine predominantly by erosion. Thus, the caffeine release closely follows the rate of erosion. This mechanism is less prone to variation between different types of active ingredient as opposed where release is mostly controlled by diffusion.

In one embodiment, the matrix uniform dispersion is prepared from a melt dispersion. Such uniform dispersion may be prepared from a melt dispersion of components i., ii., iii., at a temperature above the melting point of the PEO/HPMC (AS) mixture thus obtaining a liquid and subsequently cooling it for form a solid.

The inventors have shown that compositions of the present disclosure produced either by direct compression of the components, or by preparation from a melt state, produce equivalent release profiles of active ingredients.

In one embodiment, the composition according to the present disclosure is compressed into a tablet.

In one embodiment, the composition compressed into a tablet is not easily penetrable by water. Sufficient compression can be achieved by methods for tablet preparation known to the skilled artisan, for example, by compression under pressures of 5 tons or higher.

In one embodiment, the composition further comprises one or more further polymers each independently selected from the group consisting of ionic, non-ionic, water-insoluble polymers, and water-soluble polymers.

### Excipients

The composition according to the present disclosure may comprise one or more additional excipients. An excipient is a pharmacologically inactive substance formulated with the active ingredient of a medication.

In one embodiment, the one or more further polymers are each independently selected from the group consisting of polysaccharides, polyacrylates and polysiloxanes and derivatives thereof.

In one embodiment, the one or more further polymers are each independently selected from the group consisting of polyethylene oxide glucomannan, galactan, glucan, polygalacturonic acid, polyhdyroxyalkanoates, polyxylane, polygalactomannans, rhamnogalacturonan, polyxyloglycan, arabinogalactan, starch, alginates, xanthan gum, carrageenan, agar, dextran, pectins, cellulose, polyvinyl alcohol, polyvinyl butyral, polyvinyl pyrrolidone, methylcellulose, ethylcellulose, hydroxypropyl cellulose, hydroxypropyl methylcellulose stearate, carboxymethyl cellulose, carbomers, polyacrylic acid, poly(methylacrylic) acid, poly(methylmethacrylate), polyhydroxybutyrate, polyhydroxyvalerate, polyhydroxyphenylvalerate, polylactic acid, polyglycolic acid, a polyacrylic amide, and derivatives or copolymers thereof.

In one embodiment, the composition according to the present disclosure comprises one or more further excipients. Said one or more excipients may act as a solid carrier, diluent, flavouring agent, solubilizer, lubricant, glidant, suspending agent, binder, filler, preservative, antiadherent, wetting agent, tablet disintegrating agent, sorbent, and/or an encapsulating/coating material.

In one embodiment, the one or more additional excipients are selected from one or more of the groups consisting of: binders, fillers, lubricants, release-controlling excipients, stabilizers, plasticizers, antioxidants and preservatives.

In one embodiment, the composition comprises a filler, such as a filler selected from the group consisting of calcium carbonate, calcium phosphates, calcium sulfate, cellulose, cellulose acetate, compressible sugar, dextrate, dextrin, dextrose, ethylcellulose, fructose, isomalt, lactitol, lactose, mannitol, magnesium carbonate, magnesium oxide, maltodextrin, microcrystalline cellulose (MCC), polydextrose, sodium alginate, sorbitol, talc and xylitol.

In one embodiment, the composition comprises a binder, such as a binder selected from the group consisting of acacia, alginic acid, carbomers, carboxymethylcellulose sodium, carrageenan, cellulose acetate phthalate, chitosan, copovidone, dextrate, dextrin, dextrose, ethylcellulose, gelatin, guar gum, hydroyethyl cellulose, hydroxyethylmethyl cellulose, hydroxypropyl cellulose, hydroxypropyl starch, hypromellose, methylcellulose, poloxamer, polydextrose, polyethylene oxide, povidone, sodium alginate, sucrose, starch, pregelatinized starch and maltodextrin.

In one embodiment, the composition comprises a lubricant, such as a lubricant selected from the group consisting of calcium stearate, glycerin monostearate, glyceryl behenate, glyceryl palmitostearate, hydrogenated castor oil, hydrogenated vegetable oil, magnesium lauryl sulfate, magnesium stearate, medium chain triglyceride, palmitic acid, polyethylene glycol, sodium lauryl sulfate, stearic acid, talc, silica and zinc stearate.

A preservative may be an antimicrobial or an antioxidant. A preservative may also be a light filter or a light blocker, such as TiO₂. In one embodiment of the present disclosure, the composition comprises one or more preservatives. In one embodiment the one or more preservatives are independently selected form the group consisting of: antimicrobials, antioxidants, a light filter or a light blocker. In one embodiment, the composition comprises a release-controlling excipient, such as a release controlling excipient selected from the group consisting of functionalized celluloses, glycerin monostearate, glyceryl monooleate, glyceryl palmitate, glyceryl behenate, hydrogenated vegetable oil, guar gum, polyvinyl alcohol, alginates, xanthan gum, carnauba wax, yellow wax, white wax, zein, carrageenan, carbomers and agar.

Any other excipients suitable for the purpose of the present invention and known to the skilled person are considered encompassed by the present invention.

In one embodiment, the composition does not comprise a lubricant.

The inventors have surprisingly shown that the compositions according to the present disclosure do not require the presence of a lubricant. Lubricants are generally required to improve the properties of a powder during processing of formulations, and work by reducing friction. Due to the properties of PEO and the amounts of PEO used, a lubricant is not necessary during processing to prepare the compositions according to the present disclosure. For example, the composition does not comprise lubricants selected from: metallic salts of fatty acids, such as divalent salts of fatty acids, for example magnesium, calcium or zinc salts of fatty acids; fatty acids, fatty acids esters or talc.

### Dosage Forms

In one embodiment, the composition is a solid dosage form, such as an orally available solid dosage form. In one embodiment, the composition is a single-unit oral dosage form.

In a preferred embodiment, the composition is in the form of a tablet. For example, the matrix comprising or consisting of a uniform dispersion of caffeine, or a pharmaceutically acceptable salt thereof, or food safe salt thereof (component a); HPMC and/or HPMC-AS (component b), and PEO (component c) as described herein is in the form of a tablet.

A tablet is a pharmaceutical dosage form comprising a mixture of (an) active substance(s) and excipients, pressed or compacted into a solid dose. Tablets are simple and convenient to use. They provide an accurately measured dosage of the active ingredient(s) in a convenient portable package. Manufacturing processes and techniques can provide tablets with special properties, for example, extended release or fast dissolving formulations. Tablets are easy to weigh out and have high physical integrity.

Tablets can be manufactured by methods known in the art, for example but not limited to: vacuum compression moulding (VCM), wet granulation, dry granulation, direct compression (DCT), hot melt extrusion and calendering, roller compaction or combinations thereof. In one embodiment, the tablet is prepared as described herein.

In one embodiment, the composition is selected from the group consisting of a tablet, a mini-tablet and a micro-tablet.

In one embodiment, the composition is a monolithical tablet.

In one embodiment, the composition is a monolithical cylindrical tablet.

In one embodiment, the composition is a tablet having a total weight of 200 to 1500 mg, such as 200 to 1000 mg, 200 to 800 mg, such as 400 to 600 mg, such as 450 to 550 mg, such as 500 mg.

In one embodiment, the composition is a tablet having a total weight of 1500 mg, 1000 mg, 800 mg, or 500 mg.

In one embodiment, the composition is a tablet having a total weight of 1000 mg.

In one embodiment, the composition is a tablet having a total weight of 1000 mg.

In one embodiment, the composition is a tablet has a total weight of 500 mg.

In one embodiment, the tablet has a volume/weight ratio between 100 and 200, such as 130 to 180, for example 150 to 170, such as 160.

In one embodiment, the cylindrical tablet has one dimension measuring 0.5 to 4 mm, such as 1, 2, 3 or 4 mm and another measuring 7 to 11 mm, such as 8, 9, or 10 mm.

Thus, in one embodiment, the composition is a tablet as described herein, comprising a matrix, said matrix comprising or consisting of:
a. caffeine or a pharmaceutically acceptable salt thereof as described herein,
b. HPMC and/or HPMC-AS; and
c. polyethylene oxide (PEO), wherein the weight ratio of PEO (component c) to HPMC and/or HPMC-AS (component b) is between 8:2 and 9:0.1.

In one embodiment, the tablet comprises matrix with a weight ratio between PEO (component c) and the HPMC and/or HPMC-AS (component b) as described herein above. In one embodiment, the tablet comprises matrix with a weight ratio between PEO (component c) and the HPMC and/or HPMC-AS (component b) 8:2 to 9:0.1. In one embodiment, the weight ratio between PEO (component c) and the HPMC and/or HPMC-AS (component b) is between about 8:2, such as 9:1, 9.5:1, 9:0.5, 8.5:1 or 8.6:1.

In one embodiment, the tablet comprises a matrix with a weight ratio between PEO and HPMC-AS of 8:2 to 9:0.1. In one embodiment, the tablet comprises a matrix with a weight ratio between PEO and HPMC-AS of 8:2 to 9.5:0.5. In one embodiment, the tablet comprises a matrix with a weight ratio between PEO and HPMC-AS of 8:2 to 9:1.

In one embodiment, the tablet comprises a matrix with a weight ratio between PEO and HPMC-AS of 8:2 to 8.5:1.5.

In one embodiment, the tablet comprises between 50 and 400 mg of caffeine, such as 50, 100, 200, or 300 mg of caffeine. Preferably the tablet comprises between 50 and 300 mg of caffeine, more preferably 100 mg to 200 mg of caffeine.

In one embodiment, the tablet further comprises a coating.

A coating may be formed by materials such that it provides protection and stabilization of the formulation. The coating may comprise further excipients as described herein. The coating may be an enteric coating made from known materials in the field, that mask the bitter taste of the composition and make it easier to swallow. Techniques to include coating in tablets are known to the skilled person such as dipping in a solution of the coating polymer(s) in an organic solvent and allowing the solvent to dry.

In one embodiment, the coating material comprises one or more polymers.

In one embodiment, the coating material comprises one or more polymers selected from the group consisting of: acrylates, cellulose, and derivatives thereof.

In one embodiment, the coating material comprises a methacrylic acid-methyl methacrylate copolymer. Methacrylic acid-methyl methacrylate copolymers as coatings are common sold under brand name EUDRAGIT®. In one embodiment, the coating material comprises a methacrylic acid-methyl methacrylate copolymer with CAS number 25086-15-1.

In one embodiment, the one or more polymers of the coating material is methacrylic acid-methyl methacrylate copolymer wherein the ratio of methacrylic acid monomers and methyl methacrylate monomers is 1:1 to 1:2.

In one embodiment, the one or more polymers of the coating material is methacrylic acid-methyl methacrylate copolymer wherein the ratio of methacrylic acid monomers and methyl methacrylate monomers is 1:1.

In one embodiment, the weight-average molecular weight (Mw) of the methacrylic acid-methyl methacrylate copolymer of the coating is of about 125,000 g/moL

The inventors have found that the tablets having a coating of methacrylic acid-methyl methacrylate copolymers (EUDRAGIT®) present delayed onset of release until they reach the stomach, preventing or reducing any burst release often seen in controlled release formulations.

In another embodiment, the coating material comprises one or more of the group consisting of polyacrylates, polyacrylate derivatives and copolymers thereof.

In another embodiment, the coating material comprises one or more cellulose derivatives. In another embodiment, the coating material comprises shellac.

The coating material may comprise further excipients known in the art and commonly used in the preparation of coatings for tablets, such as binders, film formers, or plasticizers.

In one embodiment, the coating comprises a plasticizer. Known plasticizers to use in tablet coating made of polymeric materials described herein are known to the skilled person. In one embodiment, the coating material comprises triethyl citrate.

In one embodiment, the coating material comprises a polymeric material as described herein and a plasticizer in weight ratio of 5:1 to 3:1, such as 4:1.

In one embodiment, the coating comprises a methacrylic acid-methyl methacrylate copolymer as described herein, and triethyl citrate in a ratio of 4:1.

In one embodiment, the composition is a multiple-unit oral dosage form, such as granules or pellets.

In one embodiment, the composition is contained within a capsule, such as a hard shell capsule, such as a hard-shelled capsule further comprising an outer coating.

In a sustained release composition multiple factors will potentially impact the release rates of the active ingredient. The compositions of the present disclosure have been shown to provide advantageous sustained release of caffeine. The release rates may be determined by evaluating the dissolution profiles of the produced batches. In vitro drug dissolution data generated from dissolution testing experiments can be related to in vivo pharmacokinetic data by means of in vitro-in vivo correlations (IVIVC).

In one embodiment, the composition is a sustained-release composition.

As demonstrated by the examples, the formulations according to the present disclosure provide sustained release of caffeine in healthy individuals with safe return to baseline. Thus, the formulations according to the present disclosure have great potential to provide caffeine according to the subjects' needs, leading to better outcomes, reduction of side-effects and/or improved quality of life-specially for subjects requiring repeated intake.

The dissolution release rate may be determined by known methods in the art, such as for example in a USP-II apparatus under conditions of pH 6.8 and 37° C. simulating intestinal fluid.

In one embodiment, the composition provides sustained release at a rate of 100% release within 2 hours as determined in a USP-II apparatus at pH 6.8 and 37° C. simulating intestinal fluid.

### A Method of Preparation

One aspect of the present disclosure provides for a method of manufacturing a composition comprising the steps of:
a. preparing a melt uniform dispersion comprising i) PEO, ii) hydroxypropyl methyl cellulose and/or HPMC-AS and iii) an active ingredient or a pharmaceutically acceptable salt thereof, and
b. cooling the melt to obtain a solid composition.

A melt can be prepared by heating a mixture of powders to a temperature at least partially melting the powders, or one of the powders. In one embodiment, step a) comprises heating the mixture to a temperature at least partially melting the powders, or one of the powders. In one embodiment, step a) further comprises compressing the mixture.

One aspect of the present disclosure provides for a method of manufacturing a composition comprising the steps of:
(a) mixing powders of caffeine or a pharmaceutically acceptable salt thereof, HPMC and/or HPMC-AS; and PEO, and
(b) compacting the mixture to obtain uniform dispersion of components in a matrix.

The processes and methodologies to achieve uniform dispersions either through a melt dispersion or through mixing and compacting solids are well known to a person of skill in the art. For example, methodologies to implement the methods herein described include but are not limited to: dry granulation such as achieved by roller compaction, calendering, slugging or pneumatic dry granulation; compression moulding, such as vacuum compression moulding (VCM); direct compression (DCT); hot melt extrusion (HME); ultrasound assisted compaction; or wet granulation.

In one embodiment, step i) of compacting the mixture to obtain a uniform dispersion of components in a matrix is performed by dry granulation methods. In one embodiment, the dry granulation is performed by roller compaction.

In one embodiment, the method further comprises a step of compressing the composition into a solid composition.

In one embodiment, the method further comprises a step of compressing the composition into a tablet.

Compressing with sufficient force provides for tablets wherein the fissures between the granules are small and this reduces the rate of water penetration, ensuring proper erosion of the tablet. A person of skill in the art is aware on how to achieve proper compression to ensure slow rate of water penetration and how to optimize the force of compression according to the materials or equipment used.

In one embodiment, the method further comprises a step of coating the solid composition or tablet.

In one embodiment, coating is performed by dipping the solid composition or tablet in a solution of the suitable polymer, such as methacrylic acid-methyl methacrylate copolymer as described herein, for example EUDRAGIT®. The polymer and other excipients may be dissolved in a suitable amount as known in the art in an acceptable organic solvent and thereafter the tablets can be coated by dipping them in the solution for an amount of time and letting the solvent dry. The skilled artisan will be able to know suitable solvent systems for coating of tablets with polymers, such as methacrylic acid-methyl methacrylate copolymers or other polymers. The examples further elaborate on how the tablet of the present disclosure may be coated by dip coating, variations of solvent and coating parameters can be optimized by the skilled person to achieve the desired coating amount on the tablet.

In one embodiment, the coating is performed by spray drying in a fluid bed.

In one embodiment, the composition, the PEO, the HPMC and/or HPMC-AS and the caffeine or pharmaceutically acceptable salt thereof are as described herein.

In one embodiment, the composition is obtained by the method as described herein.

### Methods

One aspect of the present disclosure provides for a method for obtaining tₘₐₓ of caffeine between 4 h and 9 h in a subject, such as 5 h, such as 6 h, such as 7 h, such as 8 h, said method comprising administering the composition described herein, such as the tablet or the coated-tablet described herein.

In one embodiment, the method provides tₘₐₓ of caffeine of from 4 h and 8 h, such as from 4 h to 7 h, such as from 4 h to 6 h, such as from 4 h to 5 h. In one embodiment, the method provides tₘₐₓ of caffeine from 5 h to 6 h, such as from 5 h to 7 h, such as from 5 h to 8 h. In one embodiment, the method provides tₘₐₓ of caffeine from 5 h to 6 h, such as from 5 h to 7 h, such as from 5 h to 8 h. In one embodiment, the method provides tₘₐₓ of caffeine from 6 h to 7 h, such as from 6 h to 8 h.

One aspect of the present disclosure provides for a method for obtaining Cₘₐₓ of caffeine between 0.5 and 4 mg/mL in a subject, such as 1 mg/ml, such as 1.5 mg/mL, such as 2 mg/mL, such as 2.5 mg/mL, such as 3 mg/mL, such as 3.5 mg/mL, said method comprising administering the composition described herein, such as the tablet or the coated-tablet described herein.

In one embodiment the composition or the formulation is administered during food intake or after food intake, such as within 3 hours after food intake, such as within 2 hours after food intake, such as within 1 hour after food intake, such as within 30 minutes after food intake.

In one embodiment, the composition or the formulation is administered once daily. In one embodiment, the composition or the formulation is administered on an empty stomach. In one embodiment, the subject is fasted.

In one embodiment, the composition or the formulation provides an increase in tₘₐₓ of caffeine, or a pharmaceutically acceptable salt thereof, as compared to an equivalent amount of caffeine, or a pharmaceutically acceptable salt thereof, administered as an immediate release formulation.

In one embodiment, tₘₐₓ of caffeine, or a pharmaceutically acceptable salt thereof, is increased by at least about 1 hour, such as at least about 2 hours, for example at least about 3 hours, such as at least about 4 hours, such as at least about 5 hours, such as at least about 6 hours, such as at least about 7 hours as compared to an equivalent amount of caffeine, or a pharmaceutically acceptable salt thereof, administered as an immediate release formulation.

In one embodiment, the composition provides a Cₘₐₓ of caffeine, or a pharmaceutically acceptable salt thereof, between 0.5 and 3 μg/dL, such as between 1 and 2.5 μg/dL.

In some embodiments, the Cₘₐₓ of caffeine is of at least 0.5 μg/dL.

As referred herein the Cₘₐₓ and/or tₘₐₓ may be in blood, serum, plasma, or saliva and may be determined by known methods in the art. The examples and references provided herein exemplify how the Cₘₐₓ or tₘₐₓ may be determined.

As referred herein the Cₘₐₓ and/or tₘₐₓ may be the average or median obtained in a group of subjects, for example in a group of adult humans. In some embodiments, it may refer to the average or median Cₘₐₓ or tₘₐₓ in a group of adult humans in a fasted state.

One aspect of the present disclosure provides for a method of reducing caffeine side effects in a subject, said method comprising administering the composition described herein.

In one embodiment, the side effect is a sleep disorder, feeling of nervousness, headaches, light-headedness, urgency to urinate and/or “jitters”. “Jitters” produced by caffeine refer to feelings of anxiety, nervousness and shakiness upon consumption of caffeine.

One aspect of the present disclosure provides for a method of reducing tiredness in a subject, said method comprising administering the composition described herein.

One aspect of the present disclosure provides for a method of improving cognitive performance, energy, focus, alertness, endurance and/or mental status in a subject, said method comprising administering the composition described herein.

### EXAMPLES

### Example 1: Formulation Manufacturing and Sustained Release

### Aim

To illustrate the preparation of sustained release caffeine formulations.

### Materials and Methods

### Manufacturing of Controlled Release Caffeine Tablets (SR-321)

Caffeine (Caffeine anhydrous (BP, Ph. EUR, pharma grade, PanReac AppliChem, 142833.1608), PEO (SENTRY™ POLYOX™ WSR N80-LEO NF Grade, Dupont, excipient, GMP grade), and HPMC-AS (Shin-Etsu AQOAT® AS-LF, excipient, Harke pharma CAS number 71138-97-1, 3 mPa·s, acetyl content: 5.0-9.0%, succinyl content: 14.0-18.0%, methoxy content: 20.0-24.0%, hydroxypropyl content: 5.0-9.0%) were mixed in adequate amounts using by direct granulation and roller compaction and pressed into cylindrical tablets using a hydraulic press equipped with a conventional set of punches. The resulting un-coated tablets had the composition shown in Table 1.

TABLE 1
Tablet formulations prepared (un-coated).

| | |
|---|---|
| Caffeine | Matrix components (mg) |

| Size, weight | (mg) | PEO | Aqoat ® |
|---|---|---|---|
| 9 × 10 mm, | 100 | 360 | 40 |
| 500 mg | | | |

In addition, each tablet was coated using methacrylic acid-methyl methacrylate copolymer ((1:1), EUDRAGIT® CAS #25086-15-1, Ph. Eur. NF) by dipping in a solution of the coating material.

For the coating, a tablet was dipped two thirds into a coating solution for 15 sec, turned and allowed to dry for 30 min. The tablet was then flipped in the opposite direction and dipped two thirds into the coating solution for 15 sec, turned and allowed to dry for 30 min. This process was then repeated once more to ensure a sufficient coating layer and allowed to dry for 24 h. The coating was done at ambient temperature.

### Coating Solution

| | | |
|---|---|---|
| Eudragit L 100 (coating), 1:1 ratio | 12.0% | w/w |
| methyacrylic acid:methyl | | |
| methacrylate | | |
| Triethyl citrate (plasticizer) | 3.0% | w/w |
| Ethanol 96% (volatile solvent) | 85.0% | w/w |

The weight gain of the tablets upon coating was ˜25 mg yielding a thickness of the coat of ˜50 μm.

### Control Tablets (IR-321)

For control, an immediate release caffeine tablet was used (Cofi-Tabs, 100 mg, Vitabalans OY, article number 222276).

### Caffeine Release Study In-Vitro

The dissolution experiment was conducted in a USP-II apparatus with special inserts and 250-mL vessels (Erweka DT 70, Heusenstamm, Germany).

2 liters of 0.1 M phosphate buffer pH 6.8 was prepared. The USP bath was preheated. 250 ml 0.1 M phosphate buffer pH 6.8 was added to each of the six dissolution vessels. The vessels were then allowed to reach 37 degrees (15 min). FaSSGF (Fasted State Simulated Gastric Fluid) media was added to the beakers and simulated intestinal fluid and pH6.8 with phosphate buffer simulated intestinal fluid. The dissolution experiments were conducted at 37±0.1° C. paddle stirring rate of 50 rpm for up to 24 hrs at pH6.8 (2 of each formulation). Samples were removed at time is taken from each vessel at specific times and placed directly in a HPLC vial.

A high-performance liquid chromatograph (HPLC) method was used for quantification of caffeine using a UV-detector and a linear standard calibration curve.

### Results

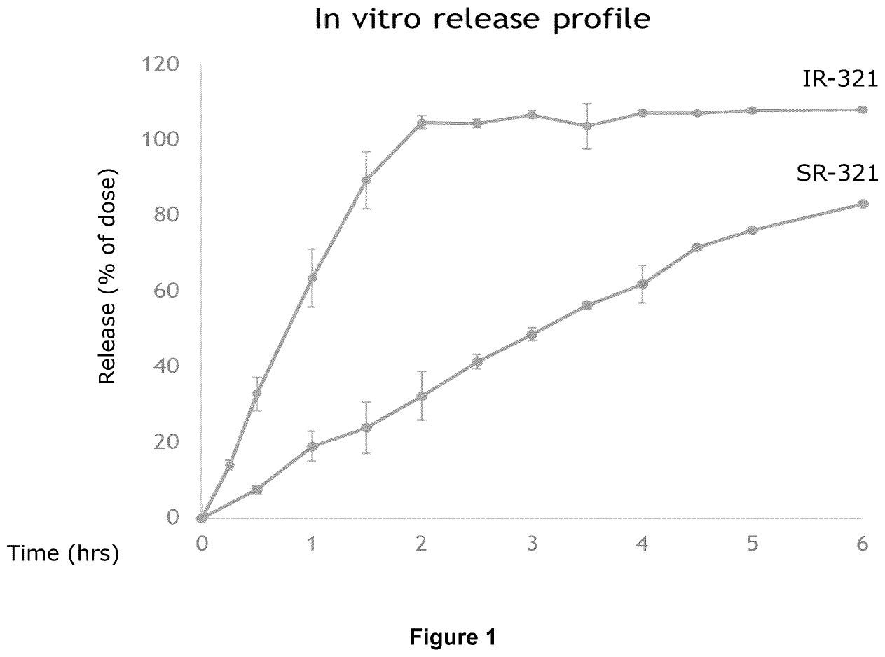

The in vitro dissolution rate of caffeine in sustained release (SR-321) and immediate release samples (IR-321) are shown in FIG. 1. The results show that the sustained release tablets released caffeine at a homogeneous rate that was slower than the immediate release samples.

### Conclusion

Compositions according to the present disclosure provide sustained release of caffeine.

### Example 2: Study in Healthy Adults

### Aim

To study the release of caffeine upon oral administration of compositions according to the present disclosure compared to an immediate release control.

### Materials and Methods

### Study in Healthy Volunteers

The study has a non-randomized, non-blinded, crossover design with two arms: immediate release caffeine and controlled release caffeine. Each participant will serve as their own control, undergoing both conditions.
Tablet (arm) 1:6 healthy subjects will be given one dose of 100 mg caffeine contained in a controlled release tablet described in Example 1.
Tablet (arm) 2:6 healthy subjects will be given one dose of 100 mg caffeine contained in an immediate release tablet (Cofi-Tabs).

### Biological Samples

0.5-1 mL saliva will be collected at 9 time points from each subject and sent to a contract laboratory for analysis of caffeine content (by HPLC-UV). The samples will be analyzed immediately (<5-7 days after sampling). The samples will be destroyed as soon as possible after analysis. The samples taken during the study will not be stored in any research biobank, nor will samples from an existing biobank be used.

The study was conducted in healthy adults aged ≥18 and ≤75 years.

Inclusion criteria: Written informed consent. Healthy adult volunteers aged ≥18 and ≤75 years. Having a BMI within the range of 18.5-35 kg/m². Capability to meet the requirements of the study and willingness to adhere to the protocol (visits, sampling, etc.).

Exclusion criteria: Pregnant or breastfeeding individual (to be confirmed verbally. If pregnancy cannot be excluded, a pregnancy test will be offered).

Cardiovascular or metabolic disorders. Caffeine sensitivity or intolerance. Allergic to caffeine or related compounds. Substance abuse or alcohol dependence. Regular use of tobacco or nicotine containing products Any condition that in the judgement of the investigator may place the subject at an increased risk or affect the quality of the study data (e.g., gastrointestinal disorders, concomitant medications).

### Meals and Dietary Restrictions:

The subject must refrain from drinking any caffeine containing liquids (e.g., coffee, Coca Cola, energy drinks, chocolate) at least 24 hours prior to administration.
The subject must refrain from eating for at least 12 hours prior to administration.
The subject must refrain from drinking water at least 2 hours prior to administration.
Post administration, the subject can drink non-caffeine containing liquids as normal.
A caffeine-free meal will be served approximately 6 hours post administration.
The subjects must refrain from drinking and eating any caffeine containing liquids or food until the final sampling point (24 hours post dose).

### Risk Assessment

Caffeine is classified by the US Food and Drug Administration (FDA) as generally recognized as safe (Blanchard et al. 1983). Toxic doses of over 10 grams per day for an adult are much higher than the typical dose of under 500 milligrams per day. The European Food Safety Authority reported that up to 400 mg of caffeine per day (around 5.7 mg/kg of body mass per day) does not raise safety concerns for non-pregnant adults (21 Code of Federal Regulations, CFR). Caffeine is rapidly absorbed after oral administration, reaching peak plasma concentrations within 30 minutes to 2 hours after administration. Bioavailability reaches around 100% in humans.

In this study, a dose of 100 mg caffeine is administered to the subjects once. This dose is comparable to a normal cup of coffee or to the content in a common energy drink and is significantly lower than the 400 mg of caffeine per day reported safe by the European Food safety Authority.

Study treatment procedures include oral intake of one tablet and sampling of saliva (non-invasive sampling, expectorating into a tube) are not considered to cause any pain or in any way negatively affect the subjects. The tablet has been designed for easy administration.

Based on the above, the risk for adverse events, or other negative consequences for the subjects is considered low.

### In Vitro In Vivo Correlations (IVIVC) of Caffeine

The in vitro in vivo correlation (IVIVC) method uses in vitro dissolution data to derive blood drug levels using pharmacokinetic parameters of a test product. The convolution approach starts with dissolution results or profiles and develops the in vivo or drug concentration-time estimated profiles. Thus, using the dissolution rate data obtained in Example 1, the expected profiles of caffeine were modelled using a well-validated method as described in Qureshi et al. 2010. Briefly these profiles were converted into discrete dosage segments, the bioavailability of caffeine is 100%, half-life (t½) is estimated to be 7.5 h after baseline correction (subtracting concentration at time 0), volume of distribution (Vd) is 0.8 L/kg for a body weight (BW) of 66 kg individual. Then, the different pharmacokinetic parameters were calculated.

### Results

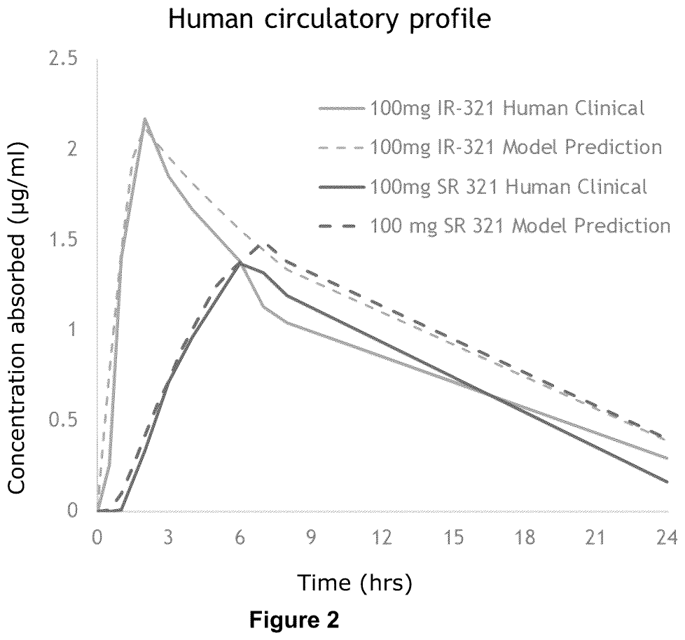

The absorption from the modified release formulation (SR 321) was compared with an immediate release marketed formulation (IR-321). The modelled absorption data based in vitro dissolution was compared with the actual in vivo data (n=6) and demonstrated a strong prediction (FIG. 2).

Preliminary calculations of the pharmacokinetic data based on the caffeine measurements samples from human subjects were performed and show similar area under the curve (AUC) values for IR vs the modified release platform, whereas the Cₘₐₓ appears to be lower for the modified release platform owing to the delayed tₘₐₓ (Table 2).

TABLE 2
Pharmacokinetic parameters measured in human subjects

| | Sustained release | Immediate release | |
|---|---|---|---|
| Parameter | tablet | control | P value |
| Cₘₐₓ (mg/mL) | 1.48 | 2.17 | <0.05 |
| tₘₐₓ (h) | 5.75 | 1.75 | <0.05 |
| AUC (mg · h · mL⁻¹) | 18.81 | 23.42 | >0.05 |

Statistical analysis was done with a t-test paired two sample for means.

### Conclusion

Compositions according to the present disclosure provide sustained release of caffeine, with a tₘₐₓ of 5.75 h, compared to 1.75 h for immediate release caffeine compositions.

### REFERENCES

21 Code of Federal Regulations (CFR) 182.1180. https://www.ecfr.gov/current/title-21/chapter-I/subchapter-B/part-182/subpart-B/section-182.1180. Accessed 2024 Oct. 17.
Blanchard J, Sawers S J. 1983. The absolute bioavailability of caffeine in man. Eur J Clin Pharmacol. 1983; 24 (1): 93-8. doi: 10.1007/BF00613933.
Herrick J., Shecterle L. M. and Cyr J. A. St. 2009. D-ribose—An additive with caffeine,
Medical Hypotheses, Volume 72, Issue 5, Pages 499-500. doi.org/10.1016/j.mehy.2008.12.038.
Mclellan T M, Caldwell J A, Lieberman H R. 2016. A review of caffeine's effects on cognitive, physical and occupational performance. Neurosci Biobehav Rev. 71:294-312. doi: 10.1016/j.neubiorev.2016.09.001.
Perera et al., Pharmacokinetics of caffeine in plasma and saliva, and the influence of caffeine abstinence on CYP1A2 metrics, J Pharm Pharmacol, 2011, 63:1161-1168
Qureshi S A. In Vitro-In Vivo Correlation (IVIVC) and Determining Drug Concentrations in Blood from Dissolution Testing—A Simple and Practical Approach. The Open Drug Delivery Journal, 2010, Volume 4

1-61. (canceled)

62. A composition comprising:
(a) caffeine, or a pharmaceutically acceptable salt thereof, or food safe salt thereof;
(b) hydroxypropyl methyl cellulose acetate succinate (HPMC-AS) and/or hydroxypropyl methyl cellulose (HPMC), and
(c) polyethylene oxide (PEO);
wherein the weight ratio between PEO (c) and the HPMC or HPMC-AS (b) is of 8:2 to 9:0.1.

63. The composition according to claim 62, wherein the weight ratio between PEO (c) and the HPMC and/or HPMC-AS (b) is from 9:1 to 9.5:0.5.

64. The composition according to claim 62, wherein caffeine, or a pharmaceutically acceptable salt thereof, is present in an amount of less than 50% by weight of the composition.

65. The composition according to claim 62, wherein the HPMC-AS and/or hydroxypropyl methyl cellulose is present in an amount of about 1% to 20% by weight of the composition.

66. The composition according to claim 62, wherein the PEO has an average molecular weight between 10 kDa to 2000 kDa.

67. The composition according to claim 62, wherein PEO is present in an amount of about 50 to 99% by weight of the composition.

68. The composition according to claim 62, wherein the composition is a uniform dispersion of caffeine, or a pharmaceutically acceptable salt thereof; the HPMC and/or HPMC-AS; and the PEO.

69. The composition according to claim 62, further comprising one or more polymers each independently selected from the group consisting of polyethylene oxide glucomannan, galactan, glucan, polygalacturonic acid, polyhdyroxyalkanoates, polyxylane, polygalactomannans, rhamnogalacturonan, polyxyloglycan, arabinogalactan, starch, alginates, xanthan gum, carrageenan, agar, dextran, pectins, cellulose, polyvinyl alcohol, polyvinyl butyral, polyvinyl pyrrolidone, methylcellulose, ethylcellulose, hydroxypropyl cellulose, hydroxypropyl methylcellulose stearate, carboxymethyl cellulose, carbomers, polyacrylic acid, poly(methylacrylic) acid, poly(methylmethacrylate), polyhydroxybutyrate, polyhydroxyvalerate, polyhydroxyphenylvalerate, polylactic acid, polyglycolic acid, a polyacrylic amide, and derivatives or copolymers thereof.

70. The composition according to claim 62, further comprising one or more additional excipients selected from the group consisting of: binders, fillers, lubricants, release-controlling excipients, stabilizers, antioxidants and preservatives.

71. The composition according to claim 62, wherein the composition is a solid dosage form, such as a single-unit oral dosage form, or a multiple-unit oral dosage form.

72. The composition according to claim 62, wherein the composition is a tablet, a mini-tablet or a micro-tablet.

73. The composition according to claim 72, wherein the tablet comprises from 50 to 400 mg of caffeine.

74. The composition according to claim 72, further comprising a coating.

75. The composition according to claim 74, wherein the coating comprises polyacrylate, cellulose, or derivatives thereof.

76. The composition according to claim 74, wherein the coating comprises a methacrylic acid-methyl methacrylate copolymer.

77. The composition according to claim 62, wherein the composition is a sustained-release composition.

78. A method for obtaining:
a) tₘₐₓ of caffeine of from 4 h to 9 h in a subject, said method comprising administering a composition according to claim 62 to said subject; and/or,
b) Cₘₐₓ of caffeine from 0.5 to 4 μg/mL in a subject, said method comprising administering a composition according to claim 62 to said subject.

79. The method according to claim 78, wherein the composition is:
a) administered during food intake or after food intake,
b) administered once daily, or
c) administered on an empty stomach.

80. The method according to claim 78, wherein the composition provides a Cₘₐₓ of caffeine from 1 to 2.5 μg/dL in said subject.

81. A method of improving cognitive performance, energy, focus, alertness, or endurance in a subject, said method comprising administering a composition according to claim 62 to said subject.
